(19) United States
(12) Patent Application Publication
(10) Pub. No.: US 2016/0287938 A1
(43) Pub. Date: Oct. 6, 2016

(54) EXERCISE-EFFECT DETERMINING METHOD, EXERCISE-EFFECT DETERMINING SYSTEM, AND EXERCISE-EFFECT DETERMINING DEVICE

(71) Applicant: SEIKO EPSON CORPORATION, Tokyo (JP)

(72) Inventor: Hideo SASAHARA, Chino-shi (JP)

(21) Appl. No.: 15/089,337

(22) Filed: Apr. 1, 2016

(30) Foreign Application Priority Data
Apr. 3, 2015 (JP) 2015-076607

(51) Int. Cl. A63B 24/00; A61B 5/00; A61B 5/0205

(52) U.S. Cl. CPC A63B 24/0062; A61B 5/0205; A61B 5/486; A61B 5/4866; A61B 5/681; A61B 5/024

(57) ABSTRACT

An exercise-effect determining method includes measuring a pulse wave signal and a body motion signal of a user at the time when the user is performing predetermined exercise, calculating a pulse rate during exercise on the basis of the pulse wave signal and the body motion signal, obtaining, on the basis of lactate level information representing a relation between a pulse rate and a blood lactate level of the user acquired in advance and the pulse rate during exercise, an exercise-effect determination result of determination of an effect degree of the predetermined exercise contributing to physical strength of the user, and informing the user of the exercise-effect determination result.

### CROSS-REFERENCES TO RELATED APPLICATIONS

This application claims priority to Japanese Patent Application No. 2015-076607, filed Apr. 3, 2015, the entirety of which is herein incorporated by reference.

### BACKGROUND

1. Technical Field

The present invention relates to an exercise-effect determining method, an exercise-effect determining system, and an exercise-effect determining device.

2. Related Art

For example, as described in Japanese Patent No. 3804645 (Patent Literature 1), there has been known an exercise-amount measuring device including pulse-rate setting means for setting a range of a proper pulse rate during exercise on the basis of a maximum volume of oxygen consumed per minute of a user. The exercise-amount measuring device accumulates time in which a pulse rate during exercise calculated from a pulse wave measurement value and a body motion detection value during exercise of the user is within a range set by the pulse-rate setting means to thereby measure a proper exercise amount of the user.

However, even if users have the same maximum volume of oxygen consumed per minute, since proper exercise intensity for each of the users is different with respect to exercise loads of medium intensity to high intensity, it is likely that the exercise-amount measuring device described in Patent Literature 1 cannot measure exercise amounts of exercise of the medium intensity to the high intensity.

### SUMMARY

An advantage of some aspects of the invention is to solve at least a part of the problems described above, and the invention can be implemented as the following forms or application examples.

### Application Example 1

An exercise-effect determining method according to this application example includes: measuring a pulse wave signal and a body motion signal of a user at time when the user is performing predetermined exercise; calculating a pulse rate during exercise on the basis of the pulse wave signal and the body motion signal; obtaining, on the basis of lactate level information representing a relation between a pulse rate and a blood lactate level of the user acquired in advance and the pulse rate during exercise, an exercise-effect determination result of determination of an effect degree of the predetermined exercise contributing to physical strength of the user; and informing the user of the exercise-effect determination result.

For example, in the exercise-amount measuring method in the past for measuring an exercise amount using a maximum volume of oxygen consumed per minute (VO₂ₘₐₓ), since proper exercise intensity of each user is different with respect to exercise loads of medium intensity to high intensity, it is likely that measurement of exercise amounts of exercise of medium intensity to high intensity cannot be accurately measured.

According to this application example, by comparing the pulse rate during exercise at the time when the user performs the predetermined exercise and the lactate level information representing the relation between the pulse rate and the blood lactate level of the user acquired in advance, the exercise-effect determination result of the determination of the effect degree of the predetermined exercise contributing to the physical strength of the user is obtained. In this way, the lactate level information representing the relation between the pulse rate and the blood lactate level of each user includes various thresholds corresponding to an exercise load of low intensity and exercise loads of medium intensity to high intensity. By exercise evaluation using these thresholds, it is possible to perform appropriate exercise evaluation for each user with respect to exercise loads of a wide range of intensity.

Therefore, it is possible to provide the exercise-effect determining method capable of appropriately determining, according to the physical strength and an exercise purpose of the user, an effect degree indicating to which degree exercise (predetermined exercise) performed by the user contributes to the physical strength of the user.

### Application Example 2

In the exercise-effect determining method according to the application example, the lactate level information may include one of a lactate threshold (LT) and an onset of blood lactate accumulation (OBLA).

According to this application example, the lactate threshold (LT) and the onset of blood lactate accumulation (OBLA) are points where a blood lactate level starts a conspicuous rise. Both of the lactate threshold (LT) and the onset of blood lactate accumulation (OBLA) can be relatively easily measured and acquired by an existing blood lactate level measuring device as lactate level information.

The lactate threshold (LT) is a threshold applied in a range of blood lactate concentration of 1 mmol/L to 3 mmol/L. The onset of blood lactate accumulation (OBLA) indicates a point in time when the blood lactate concentration reaches 4 mmol/L. Therefore, there is an effect that it is possible to select, for each user, lactate level information corresponding to the physical strength and an exercise purpose of the user and perform more appropriate exercise effect determination.

### Application Example 3

In the exercise-effect determining method according to the application example, the lactate level information may include any one of an aerobic threshold (AeT), an anaerobic threshold (AT), and a ventilation threshold (VT).

According to this application example, all of the aerobic threshold (AeT), the anaerobic threshold (AT), and the ventilation threshold (VT) are lactate level information based on changes in a pulse rate and exhalation gas during exercise. By exercise evaluation using these thresholds, it is possible to perform appropriate exercise evaluation for each user with respect to exercise loads of a wide range of intensity.

The aerobic threshold (AeT) and the ventilation threshold (VT) are thresholds applied in a range of blood lactate concentration of 1 mmol/L to 3 mmol/L. The anaerobic threshold (AT) indicates a point in time when the blood lactate concentration reaches 4 mmol/L. Therefore, there is an effect that it is possible to select, for each user, lactate level information corresponding to the physical strength and an exercise purpose of the user and perform more appropriate exercise effect determination.

### Application Example 4

In the exercise-effect determining method according to the application example, the exercise-effect determining method may include a plurality of kinds of the lactate level information, and the user may be able to set and change the lactate level information according to a purpose.

According to this application example, there is an effect that it is possible to select, for each user, lactate level information corresponding to the physical strength and an exercise purpose of the user and perform more appropriate exercise effect determination.

### Application Example 5

An exercise-effect determining system according to this application example includes: a lactate level information data table in which lactate level information representing a relation between a pulse rate and a blood lactate value of a user is stored; a pulse wave measurer configured to measure a pulse wave signal of the user at time when the user is performing predetermined exercise; a body motion detector configured to detect a body motion signal of the user at the time when the user is performing the predetermined exercise; a pulse rate calculator configured to calculate a pulse rate during exercise on the basis of the pulse wave signal and the body motion signal; an exercise effect determiner configured to obtain, on the basis of a comparison result of the lactate level information of the lactate level information data table and the lactate level information corresponding to the pulse rate during exercise, an exercise-effect determination result of determination of an effect degree of the exercise intensity of the predetermined exercise contributing to physical strength of the user; and an informing unit configured to inform the user of the exercise-effect determination result.

According to this application example, by comparing the pulse rate during exercise calculated on the basis of the pulse wave signal and the body motion signal at the time when the user performs the predetermined exercise and the lactate level information representing the relation between the pulse rate and the blood lactate level of the user acquired in advance, the exercise-effect determination result of the determination of the effect degree of the predetermined exercise contributing to the physical strength of the user is obtained. In this way, the lactate level information representing the relation between the pulse rate and the blood lactate level of each user includes various thresholds corresponding to an exercise load of low intensity and exercise loads of medium intensity to high intensity. By exercise evaluation using these thresholds, it is possible to perform appropriate exercise evaluation for each user with respect to exercise loads of a wide range of intensity.

Therefore, it is possible to provide the exercise-effect determining system capable of appropriately determining, according to the physical strength and an exercise purpose of the user, an effect degree indicating to which degree exercise (predetermined exercise) performed by the user contributes to the physical strength of the user.

### Application Example 6

In the exercise-effect determining system according to the application example, the exercise-effect determining system may further include a determination mode setter with which the user can set and change the lactate level information according to a purpose.

According to this application example, there is an effect that it is possible to select and set, for each user, with the determination mode setter, the lactate level information corresponding to the physical strength and an exercise purpose of the user and perform more appropriate exercise effect determination.

### BRIEF DESCRIPTION OF THE DRAWINGS

The invention will be described with reference to the accompanying drawings, wherein like numbers reference like elements.

FIG. 1 is an explanatory diagram showing a configuration example of an exercise-effect determining system according to a first embodiment.

FIG. 2 is an explanatory diagram showing a configuration example of a system including the exercise-effect determining system shown in FIG. 1.

FIGS. 3A and 3B are explanatory diagrams showing an example of the exterior of a wearable device included in the system shown in FIG. 2 viewed from different directions.

FIG. 4 is an explanatory diagram showing an example of the exterior of the wearable device shown in FIGS. 3A and 3B viewed from another direction.

FIG. 5 is a flowchart for explaining determination processing for an exercise effect (an exercise-effect determining method) according to the first embodiment.

FIG. 6 is an explanatory diagram showing an example of a lactate level information data table used in exercise-effect determination processing according to the first embodiment.

FIG. 7 is an explanatory diagram showing an example of an exercise-effect determination result according to the first embodiment.

FIG. 8 is an explanatory diagram showing an example of a lactate level information data table used in exercise-effect determination processing in a modification.

FIG. 9 is a sectional view showing an example in the past of a biological-information measuring device according to a second embodiment.

FIG. 10 is a perspective view showing the biological-information measuring device according to the second embodiment.

FIG. 11 is a sectional view showing a biological-information measuring device according to a third embodiment.

FIG. 12 is a perspective view showing a biological-information measuring device according to a fourth embodiment.

FIG. 13 is a sectional view showing a biological-information measuring device according to a fifth embodiment.

### DESCRIPTION OF EXEMPLARY EMBODIMENTS

### First Embodiment

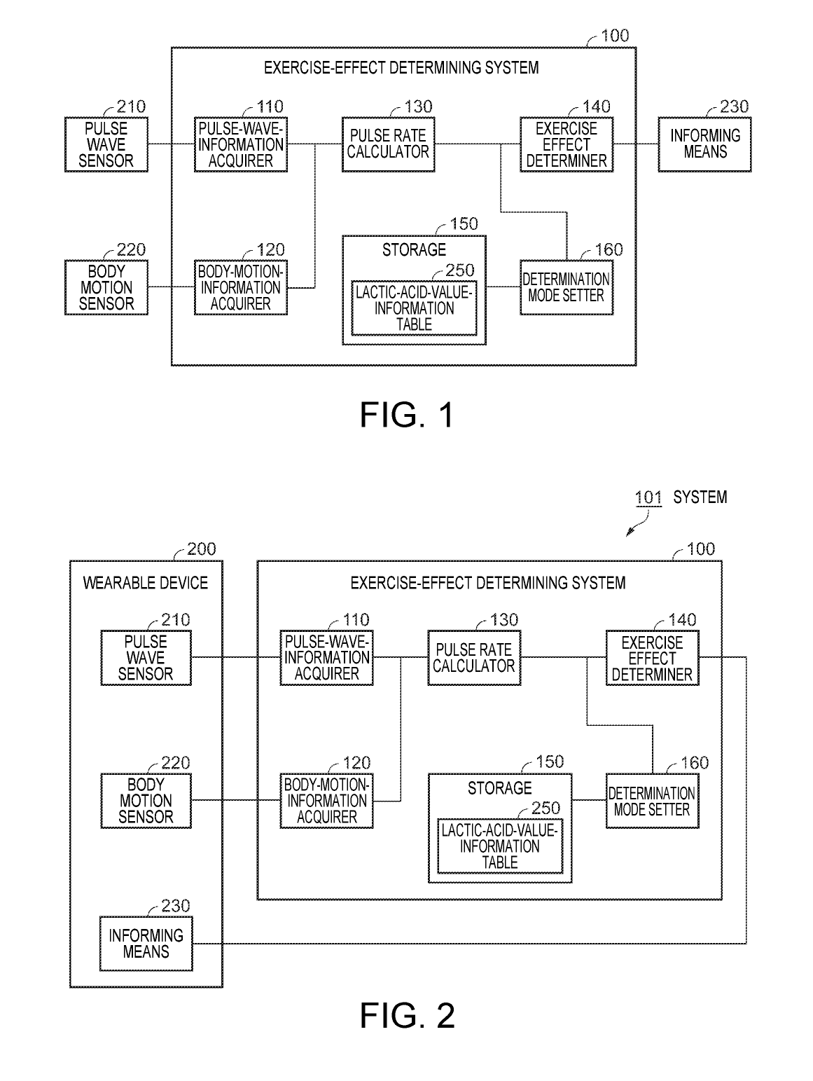

FIG. 1 shows a configuration example of an exercise-effect determining system according to a first embodiment.

First, the schematic configuration of an exercise-effect determining system 100 according to the first embodiment is explained.

As shown in FIG. 1, the exercise-effect determining system 100 according to the first embodiment includes a pulse-wave-information acquirer 110, a body-motion-information acquirer 120, a pulse rate calculator 130, a storage 150 in which a lactate level information data table 250 and the like are stored, a determination mode setter 160, an exercise effect determiner 140, and an informing unit 230.

The pulse-wave-information acquirer 110 acquires pulse wave information (a pulse wave signal) detected by a pulse wave sensor 210 functioning as a pulse wave measurer. The pulse-wave-information acquirer 110 may acquire sensor information itself of the pulse wave sensor 210 and calculate information such as a pulse wave or may acquire information (a pulse rate, etc.) calculated by another device on the basis of the sensor information. As explained in detail below, the pulse wave sensor 210 may be included or may not be included in the exercise-effect determining system 100.

The pulse wave sensor 210 is a sensor for detecting a pulse wave signal. For example, a photoelectric sensor including a light emitter and a light receiver can be used as the pulse wave sensor 210. It is known that the pulse wave sensor 210 can be realized by various sensors such as the photoelectric sensor and sensors of other forms (e.g., an ultrasound sensor). The sensors can be extensively applied to the pulse wave sensor in this embodiment.

Similarly, the body-motion-information acquirer 120 that acquires body motion information from a body motion sensor 220 functioning as a body motion detector may acquire sensor information itself of the body motion sensor 220 and calculate body motion information such as acceleration or may acquire body motion information such as acceleration calculated by another device on the basis of the sensor information. Note that, for example, a three-axis acceleration sensor can calculate acceleration values concerning respective xyz axes. Therefore, the body-motion-information acquirer 120 may perform processing for calculating one acceleration value from three acceleration values, noise reduction processing incidental to the processing, processing for calculating a moving average, and the like and may acquire information after these kinds of processing. The sensor information itself may be used as acceleration (in a broad sense, body motion information). The body motion sensor 220 may be included in or may not be included in the exercise-effect determining system 100.

The pulse rate calculator 130 calculates, on the basis of the pulse wave information (the pulse wave signal) acquired by the pulse-wave-information acquirer 110 from the pulse wave sensor 210 and the body motion information (the body motion signal) acquired by the body-motion-information acquirer 120 from the body motion sensor 220, a pulse rate during exercise at the time when a user is performing predetermined exercise.

The storage 150 has stored therein a lactate level information data table 250 in which lactate level information representing a relation between a pulse rate and a blood lactate value of the user acquired in advance is stored.

The determination mode setter 160 is a setting means configured to perform setting of a determination mode corresponding to a purpose of the user when exercise effect determination based on the pulse rate during exercise calculated by the pulse rate calculator 130 and the lactate level information stored in the lactate level information data table 250 is performed. For example, setting means of various forms such as a button, a switch, and a touch panel can be used. In an exercise-effect determining method in this embodiment, exercise effect determination is executed using a lactate threshold (LT) and an onset of blood lactate accumulation (OBLA) as the lactate level information. The determination mode setter 160 includes an LT mode and an OBLA mode.

In the determination mode set by the user with the determination mode setter 160, the exercise effect determiner 140 compares the pulse rate during exercise calculated by the pulse rate calculator 130 and the lactate level information stored in the lactate level information data table 250 to thereby determine an effect degree of the predetermined exercise performed by the user contributing to the physical strength of the user and obtain an exercise-effect determination result.

The informing unit 230 informs the user of the exercise-effect determination result of the predetermined exercise of the user obtained by the exercise effect determiner 140. As the informing unit 230, it is possible to use, for example, a display section that informs the exercise-effect determination result with characters or a diagram, a sound output section that informs the exercise-effect determination result with sound, buzzer sound, or the like, a light emitting unit configured to inform the exercise-effect determination result with a color or flashing of light, and a vibrator that informs the exercise-effect determination result by transmitting vibration to a part of the body of the user, and the like.

In FIG. 2, a detailed configuration example of a system (hereinafter referred to as exercise-effect determining system as well) 101 including the exercise-effect determining system 100 shown in FIG. 1 is shown. However, the exercise-effect determining system 101 is not limited to a configuration shown in FIG. 2. Various modified implementations such as omission of a part of components of the exercise-effect determining system 101 and addition of other components are possible. In the example of the system 101 shown in FIG. 2, a wearable device 200 includes the pulse wave sensor 210, the body motion sensor 220, and the informing unit 230. The pulse-wave-information acquirer 110 and the body-motion-information acquirer 120 of the exercise-effect determining system 100 acquire pulse wave information and body motion information from the wearable device 200. Note that the pulse wave information includes both of information (e.g., a pulse wave signal such as a pulse rate or a pulse interval) used for calculation of a pulse rate and information (e.g., sensor information) used for calculation of the information. As explained above, the pulse-wave-information acquirer 110 may acquire information of any form. The body motion information includes both of information (e.g., a body motion signal such as an acceleration value) used for determination of presence or absence of a body motion and information (e.g., sensor information) used for calculation of the information. Note that the informing unit 230 may be provided on the exercise-effect determining system 100 side rather than in the wearable device 200. The informing unit 230 may be provided in both of the wearable device 200 and the exercise-effect determining system 100 in various forms. The informing unit 230 can be set in various forms in order to more clearly inform the user of the exercise-effect determination result.

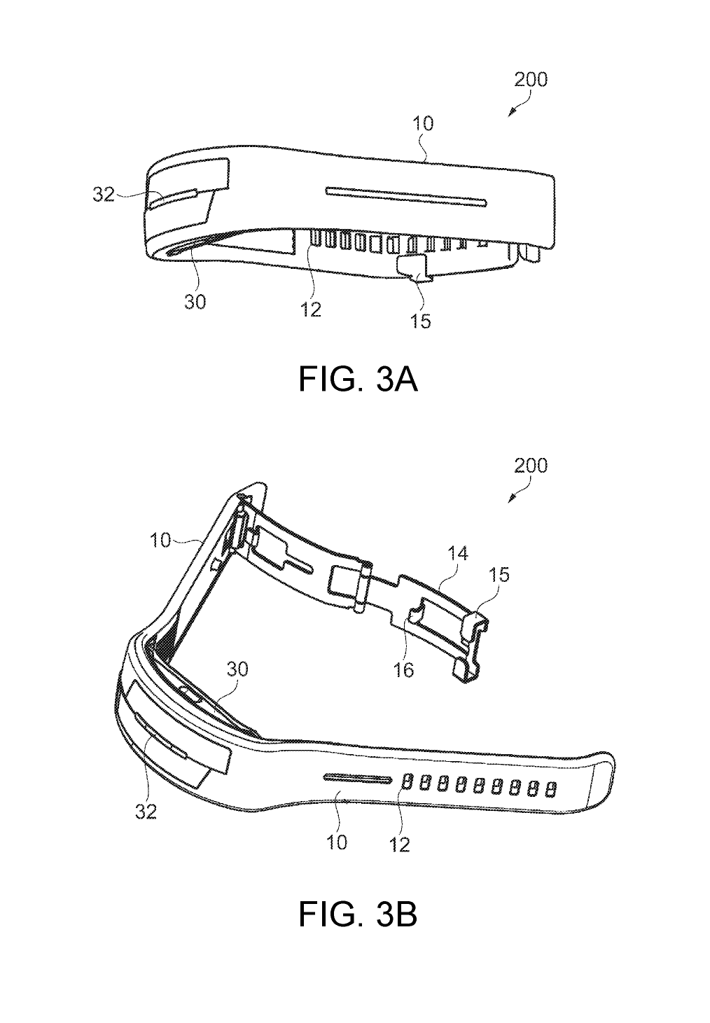

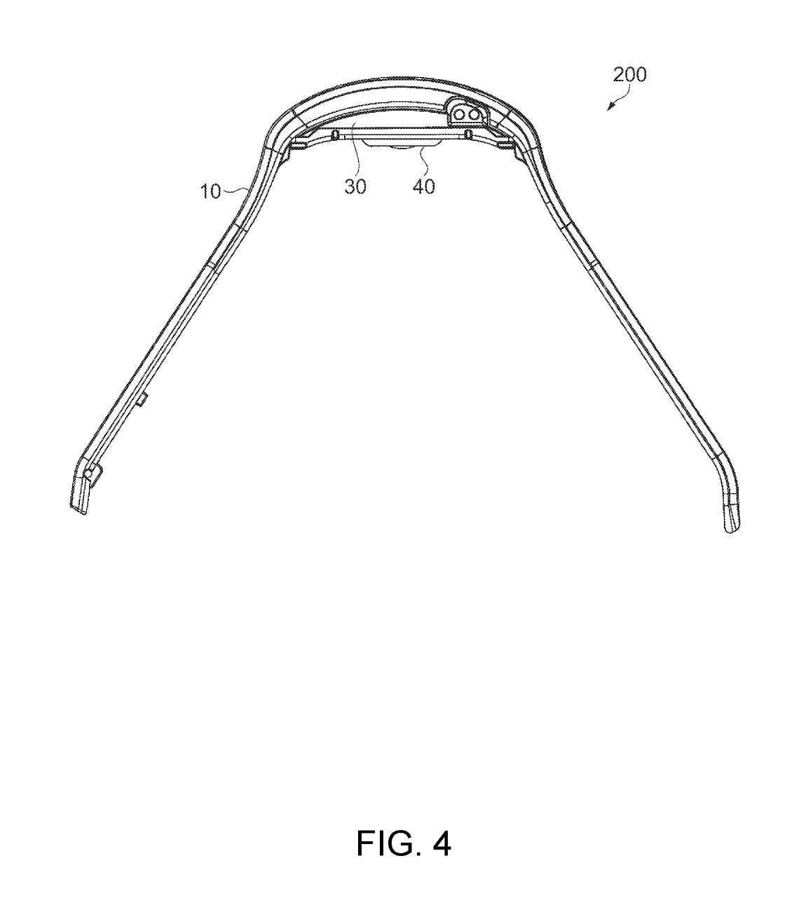

FIGS. 3A and 3B and FIG. 4 are explanatory diagrams showing examples of the exterior of the wearable device 200 included in the system 101 shown in FIG. 2 viewed from different directions. The wearable device 200 in this embodiment includes a band section 10, a case section 30, and a sensor section 40. The case section 30 is attached to the band section 10. The sensor section 40 is provided in the case section 30.

The band section 10 is a section wound around a wrist of the user to wear the wearable device 200. The band section 10 includes band holes 12 and a buckle section 14. The buckle section 14 includes a band inserting section 15 and a protrusion section 16. The user inserts one end side of the band section 10 into the band inserting section 15 of the buckle section 14 and inserts the protrusion section 16 of the buckle section 14 into the band hole 12 of the band section 10 to wear the wearable device 200 on the wrist.

The case section 30 is equivalent to a main body section of the wearable device 200. On the inside of the case section 30, various components of the wearable device 200 such as the sensor section 40 and a not-shown circuit board are provided. That is, the case section 30 is a housing that houses these components. Note that, in the sensor section 40 shown in FIGS. 3A and 3B and FIG. 4, the pulse wave sensor 210 (see FIG. 2) is disposed. The body motion sensor 220 (see FIG. 2) is disposed in the case section 30 (not shown in the figure).

A light-emitting window section 32 is provided in the case section 30. The light-emitting window section 32 is formed by a light transmitting member. In the case section 30, a light emitter functioning as an interface mounted on a flexible board is provided. Light from the light emitter is emitted to the outside of the case section 30 via the light-emitting window section 32. A color and flashing of the light by the light-emitting widow section 32 can also be used as the informing unit 230 (see FIG. 2) in this embodiment.

Details of processing of the exercise-effect determining method is explained below. The exercise-effect determining method is a method for determining, on the basis of a pulse rate during exercise at the time when the user performs predetermined exercise and lactate level information representing a relation between a pulse rate and blood lactate concentration of the user acquired in advance, an effect degree of the predetermined exercise performed by the user contributing to maintenance, improvement, and the like of the physical strength of the user.

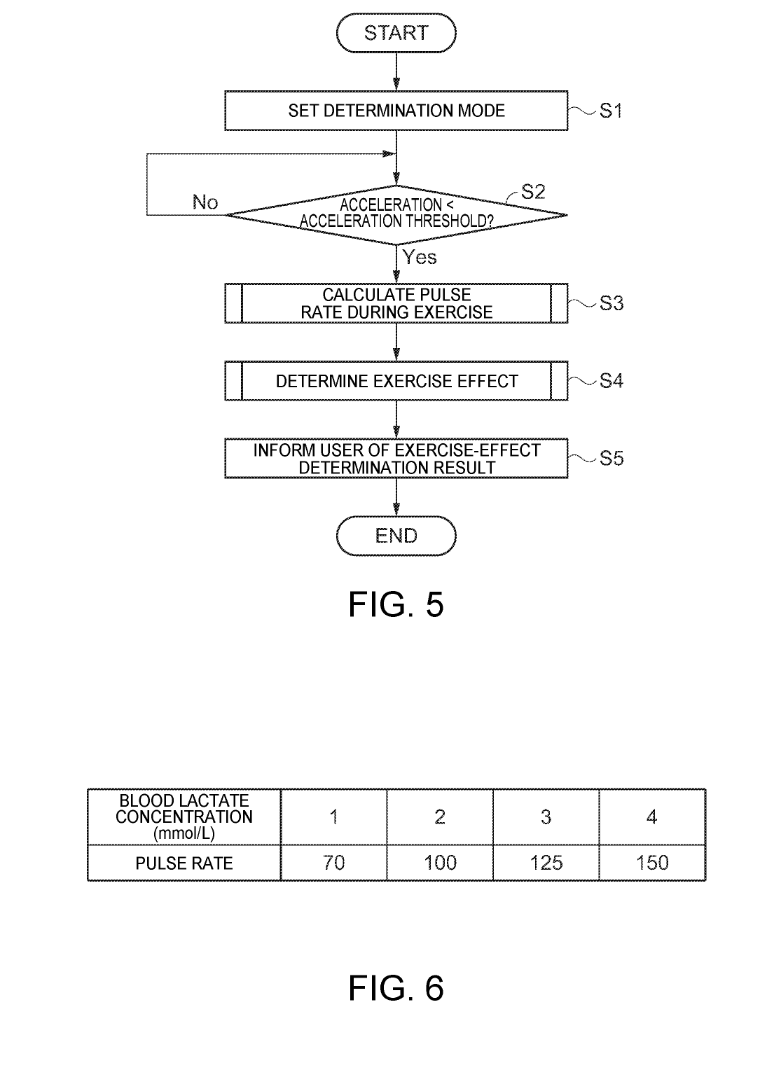

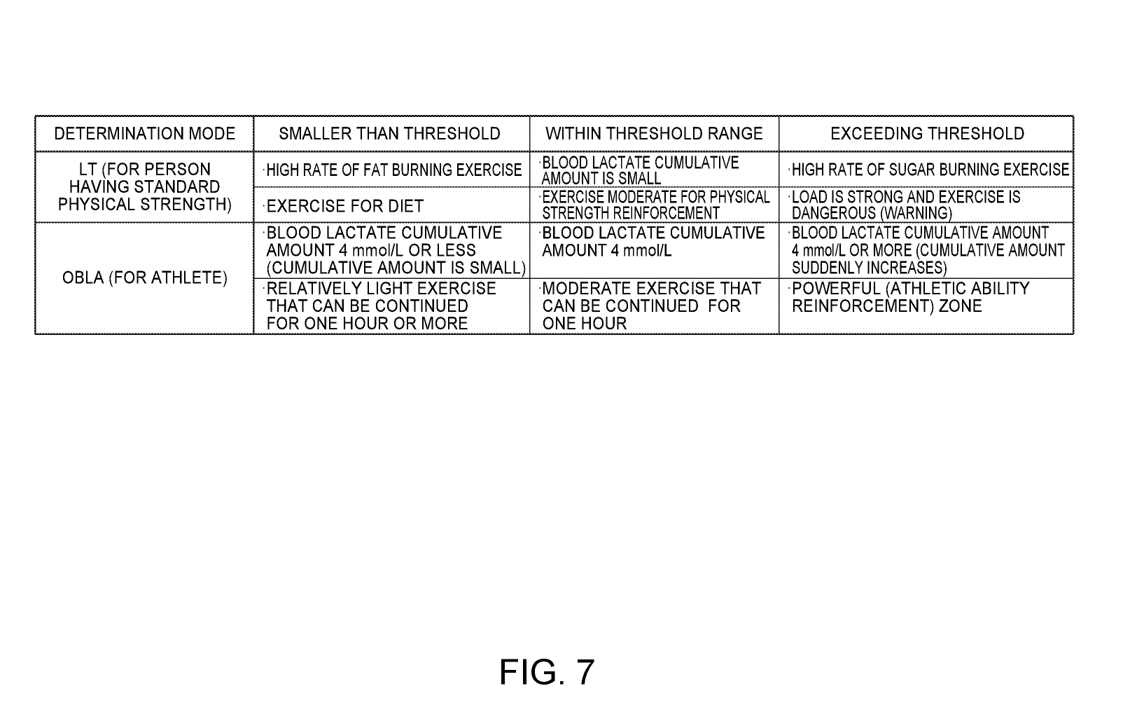

FIG. 5 is a flowchart for explaining determination processing for an exercise effect (the exercise-effect determining method) in this embodiment. FIG. 6 is an explanatory diagram showing an example of the lactate level information data table 250 used in the exercise-effect determination processing in this embodiment. FIG. 7 is an explanatory diagram showing an example of an exercise-effect determination result in this embodiment.

In the exercise-effect determining method in this embodiment, data of lactate level information representing a relation between a pulse rate and a lactic acid value during exercise of the user is acquired in advance. In the exercise effect determination in this embodiment, the LT and the OBLA are used as the lactate level information. Therefore, the LT and the OBLA during the exercise of the user are measured. In the measurement of the lactate level information, the blood lactate concentration of the user is measured using a well-known blood-lactate-concentration measuring method for measuring blood lactate concentration using a lactic acid measurement kit. The lactate level information of the user obtained by the measurement is stored in the storage 150 as the lactate level information data table 250. An example of the lactate level information data table 250 is shown in FIG. 6.

In FIG. 5, in the determination processing for an exercise effect (the exercise-effect determining method) in this embodiment, first, the user operates, with the determination mode setter 160, setting means such as a select button included in the determination mode setter 160 to thereby perform setting of a determination mode corresponding to an exercise purpose of the user (S1). As explained above, the exercise-effect determining method in this embodiment includes the LT mode and the OBLA mode. In the LT mode, exercise effect determination in a range of exercise intensity lower than exercise intensity in the OBLA mode is performed. For example, when a user having standard physical strength has a purpose of exercise such as maintenance or reinforcement of the physical strength of the user or diet, the user selects the LT mode. For example, when an athlete having physical strength higher than the standard has a purpose of maintenance and reinforcement of the physical strength, the athlete selects the OBLA mode. Note that the determination mode setting by the determination mode setter 160 only has to be performed before exercise effect determination by the exercise effect determiner 140 explained below.

Subsequently, in S2, the exercise effect determiner 140 checks whether acceleration serving as body motion information acquired by the body-motion-information acquirer 120 exceeds a predetermined threshold and determines whether the user has started the predetermined exercise. In the case of NO in S2, the exercise effect determiner 140 can determine that the user has not started the predetermined exercise. Therefore, the exercise effect determiner 140 loops the processing in S2 without particularly performing processing. On the other hand, in the case of YES in S2, the exercise effect determiner 140 determines that the user has started the predetermined exercise. Therefore, the pulse rate calculator 130 calculates a pulse rate during exercise on the basis of pulse wave information (a pulse wave signal) acquired by the pulse-wave-information acquirer 110 at the nearest timing (S3).

Subsequently, the exercise effect determiner 140 compares the pulse rate during exercise of the user calculated by the pulse rate calculator 130 and the lactate level information of the lactate level information data table 250 of the storage 150 to thereby determine an effect degree of the exercise performed by the user contributing to the physical strength of the user and obtain an exercise-effect determination result (S4).

The exercise-effect determination result of the user obtained by the exercise effect determiner 140 is informed to the user by the informing unit 230 (S5).

In FIG. 7, an example of the exercise-effect determination result in this embodiment is shown. In FIG. 7, when the determination mode is set to LT by the determination mode setting (S1), it may be considered that the user has standard physical strength and has an exercise purpose such as maintenance and reinforcement of the physical strength of the user or diet. In general, the LT (the lactate threshold) is considered to be a work threshold corresponding to exercise intensity in a low range. The LT indicates a state in which blood oxygen concentration reaches 1 mmol/L (sometimes referred to as LT1) or a state in which blood lactate concentration reaches 2 mmol/L (sometimes referred to as LT2). The LT sometimes indicates a state in which the blood lactate concentration is between 1 mmol/L and 3 mmol/L.

As a result of collating the pulse rate during exercise of the user with the lactate level information data table 250 (FIG. 6), when the pulse rate during exercise is within an LT threshold range, it can be determined that the exercise of the user is exercise suitable for physical strength reinforcement of the user having the standard physical strength in a state in which a accumulation of lactic acid in the blood is small while having exercise intensity of a fixed level. Therefore, the informing unit 230 informs the user that the exercise is, for example, “exercise moderate for physical strength reinforcement”.

As a result of collating the pulse rate during exercise of the user with the lactate level information table 250, when the pulse rate during exercise is smaller than the LT threshold, the exercise is considered to be relatively light exercise that the user having the standard physical strength can continue for a fixed time and is considered to be exercise with a high rate of fat burning exercise. The informing unit 230 informs the user that the exercise intensity is, for example, “exercise intensity for diet”.

As a result of collating the pulse rate during exercise of the user with the lactate level information table 250, when the pulse rate during exercise exceeds the LT threshold, the exercise is considered to be exercise with a high load for the user having the standard physical strength and is considered to be exercise with a high rate of sugar burning exercise. The informing unit 230 informs (warns) the user that the exercise is, for example, exercise with an excessively high load and might be dangerous.

On the other hand, when the determination mode is set to the OBLA by the determination mode setting (S1), the user may be considered to be an athlete having physical strength higher than the standard and have maintenance of the physical strength of the athlete and further physical strength reinforcement as an exercise purpose. The OBLA (the onset of blood lactate accumulation) indicates a point in time when blood oxygen concentration reaches 4 mmol/L.

As a result of collating the pulse rate during exercise of the user with the lactate level information data table 250 (FIG. 5), when the pulse rate during exercise is within an OBLA threshold range, the exercise of the user can be determined as having moderate exercise intensity still with a small lactic acid accumulation in the blood for the user (e.g., an athlete) having physical strength higher than the standard and with a high rate of sugar burning exercise and is sometimes considered to be exercise that the athlete can continue for about one hour. Therefore, the informing unit 230 informs that user that the exercise is, for example, “moderate exercise that can be continued”.

As a result of collating the pulse rate during exercise of the user with the lactate level information data table 250, when the pulse rate during exercise is smaller than the OBLA threshold (and exceeds the LT), the exercise intensity is sometimes considered to be exercise intensity with a small lactic acid accumulation in the blood for the user (e.g., an athlete) having physical strength higher than the standard and with a high rate of sugar burning exercise and the exercise is sometimes considered to be exercise that the athlete can continue for one hour or more. Therefore, the informing unit 230 informs the user that the exercise is, for example, “relatively light exercise that can be continued for one hour or more”.

As a result of collating the pulse rate during exercise of the user with the lactate level information data table 250, when the pulse rate during exercise exceeds the OBLA threshold, the exercise is considered to be exercise with a high load in which the lactic acid accumulation in the blood suddenly increases and that even the athlete cannot continue for a long time. The informing unit 230 informs the user that the exercise is, for example, exercise with a high load in a “powerful zone”, a “challenge mode”, or the like with which the athlete can achieve improvement of a level such as further record improvement or inspires the user with effective sound or the like.

As explained above, with the system (the exercise-effect determining system) 101 and the exercise-effect determining method using the system according to the first embodiment, it is possible to obtain effects explained below.

According to this embodiment, the system 101 includes the lactate level information data table (the storage) in which the lactate level information representing the relation between the pulse rate and the blood lactate value of the user is stored, the pulse wave sensor 210 that measures a pulse wave signal of the user at the time when the user is performing the predetermined exercise, and the body motion sensor 220 that detects a body motion signal of the user at the time when the user is performing the predetermined exercise. The system 101 calculates the pulse rate during exercise with the pulse rate calculator 130 on the basis of the pulse wave information measured by the pulse wave sensor 210 in the period in which the body motion sensor 220 detects that the user is performing exercise. The system 101 obtains, on the basis of the comparison result of the pulse rate during exercise and the lactate level information of the lactate level information table 250 acquired in advance, with the exercise effect determiner 140, the exercise-effect determination result of the determination of the effect degree of the exercise intensity of the predetermined exercise contributing to the physical strength of the user. The system 101 informs the user of the exercise-effect determination result with the informing unit 230.

Consequently, it is possible to solve the problem in that, for example, in the exercise-amount measuring method in the past for measuring an exercise amount using the maximum volume of oxygen consumed per minute (VO₂ₘₐₓ), since proper exercise intensity of each user is different with respect to exercise loads of medium intensity to high intensity, it is likely that measurement of exercise amounts of exercise of medium intensity to high intensity cannot be accurately measured. Specifically, the lactate level information representing the relation between the pulse rate and the blood lactate level of each user includes the various work thresholds such as the LT and the OBLA corresponding to an exercise load of low intensity and exercise loads of medium intensity to high intensity. Since the exercise evaluation is performed using these work thresholds, it is possible to perform appropriate exercise evaluation for each user with respect to exercise loads of a wide range of intensity. Therefore, it is possible to provide the exercise-effect determining method capable of appropriately determining, according to the physical strength and an exercise purpose of the user, an effect degree indicating to which degree exercise performed by the user contributes to the physical strength of the user.

The system 101 in this embodiment includes the plurality of kinds of work thresholds such as the LT and the OBLA as the threshold of the lactate level information in the lactate level information data table 250 of the storage 150 and further includes the determination mode setter 160. The user sets, with the determination mode setter 160, the threshold of the lactate level information to the LT or the OBLA according to an exercise purpose of the user and performs the exercise effect determination in a desired mode.

Consequently, it is possible to select, for each user, lactate level information corresponding to the physical strength and an exercise purpose of the user and perform more appropriate exercise effect determination.

Note that the invention is not limited to the first embodiment. It is possible to add various changes, improvements, and the like to the first embodiment. Modifications are explained below.

### Modifications

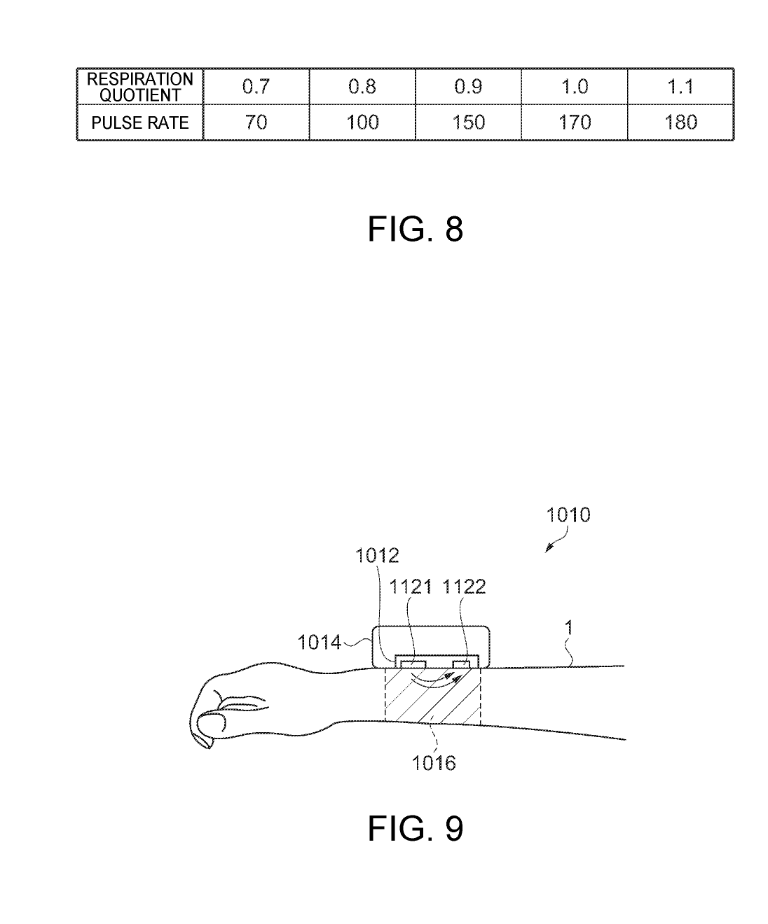

FIG. 8 is an explanatory diagram showing an example of the lactate level information data table 250 used in the exercise-effect determination processing in a modification.

In the embodiment, the LT (the lactate threshold) and the OBLA (the onset of blood lactate accumulation) are used as the lactate level information used in the exercise-effect determining method. However, the invention is not limited to this configuration.

An exercise-effect determining method according to the modification is explained. Note that components same as the components in the embodiment are denoted by the same numbers and redundant explanation of the components is omitted.

In the exercise-effect determining method in this modification, as the lactate level information, an aerobic threshold (AeT), an anaerobic threshold (AT), and a ventilation threshold (VT) representing a relation between oxygen concentration or carbon dioxide concentration in exhalation gas or an exhalation amount during exercise and a pulse rate measured by a publicly-known open circuit method during exercise of each user are used. By performing the exercise effect determination using the AeT, the AT, and the VT as lactate level information, it is possible to perform appropriate exercise effect determination for each user with respect to exercise loads of a wide range of intensity. Lactate level information of the user obtained by measurement or the like is stored in the storage 150 as the lactate level information data table 250. In FIG. 8, an example of the lactate level information data table 250 in this modification is shown. In FIG. 8, the lactate level information data table 250 in this modification represents a relation between a respiration quotient and a pulse rate during exercise of each user. The respiration quotient means a volume ratio of a carbon dioxide emission amount to an oxygen consumption amount until nutrients are decomposed in the body of the user and converted into energy in a certain time.

A flow of determination processing for an exercise effect (the exercise-effect determining method) in this modification is the same as the flow of the flowchart shown in FIG. 5. Differences in the steps of the processing flow in the first embodiment are explained below. In FIG. 5, first, the user operates setting means of the determination mode setter 160 to thereby set a determination mode corresponding to an exercise purpose of the user (S1). As explained above, the exercise-effect determining method in this modification includes the AeT mode, the AT mode, and the VT mode. The AeT mode indicates exercise intensity substantially equivalent to the exercise intensity in the LT mode in the first embodiment. In the AT mode and the VT mode, the exercise effect determination is performed in a range of exercise intensity substantially equivalent to the range of the exercise intensity in the OBLA mode in the first embodiment.

Following S1, in S2 and S3, processing same as the processing in the embodiment is performed. The pulse rate calculator 130 calculates a pulse rate during exercise on the basis of pulse wave information (a pulse wave signal) acquired by the pulse-wave-information acquirer 110 at timing when the user is performing the predetermined exercise.

Subsequently, the exercise effect determiner 140 compares the pulse rate during exercise of the user calculated by the pulse rate calculator 130 and the lactate level information of the lactate level information data table 250 to thereby determine an effect degree of the exercise performed by the user contributing to the physical strength of the user and perform the exercise effect determination (S4) and informs, with the informing unit 230, the user of a result of the exercise effect determination (S5).

The exercise-effect determination result is generally the same as the exercise-effect determination result in the embodiment shown in FIG. 7 except that the LT is replaced with the AeT and the AT and the OBLA are replaced with the AT and VT.

That is, when the determination mode is set to the AeT in the determination mode setting (S1), the user may be considered to have the standard physical strength and have maintenance and reinforcement of the physical strength of the user, diet, or the like as an exercise purpose.

As a result of collating the pulse rate during exercise of the user with the lactate level information data table 250 (FIG. 8), when the pulse rate during exercise is within an AeT threshold range, it can be determined that the exercise of the user is exercise suitable for physical strength reinforcement of the user having the standard physical strength in a state in which a accumulation of lactic acid in the blood is small while having exercise intensity of a fixed level. Therefore, the informing unit 230 informs the user that the exercise is, for example, “exercise moderate for physical strength reinforcement”.

As a result of collating the pulse rate during exercise of the user with the lactate level information table 250, when the pulse rate during exercise is smaller than the AeT threshold, the exercise is considered to be relatively light exercise that the user having the standard physical strength can continue for a fixed time and is considered to be exercise with a high rate of fat burning exercise. The informing unit 230 informs the user that the exercise intensity is, for example, “exercise intensity for diet”.

As a result of collating the pulse rate during exercise of the user with the lactate level information table 250, when the pulse rate during exercise exceeds the AeT threshold, the exercise is considered to be exercise with a high load for the user having the standard physical strength and is considered to be exercise with a high rate of sugar burning exercise. The informing unit 230 informs (warns) the user that the exercise is, for example, exercise with an excessively high load and might be dangerous.

On the other hand, when the determination mode is set to the AT or VT by the determination mode setting (S1), the user may be considered to be an athlete having physical strength higher than the standard and have maintenance of the physical strength of the athlete and further physical strength reinforcement as an exercise purpose.

That is, as a result of collating the pulse rate during exercise of the user with the lactate level information data table 250 (FIG. 8), when the pulse rate during exercise is within an AT or VT threshold range, the exercise of the user can be determined as having moderate exercise intensity still with a small lactic acid accumulation in the blood for the user (e.g., an athlete) having physical strength higher than the standard and with a high rate of sugar burning exercise, for example, exercise intensity that the athlete can continue for about one hour. Therefore, the informing unit 230 informs that user that the exercise is, for example, “moderate exercise that can be continued”.

As a result of collating the pulse rate during exercise of the user with the lactate level information data table 250, when the pulse rate during exercise is smaller than the AT or VT threshold (and exceeds the AeT), the exercise intensity can be determined as exercise intensity with a small lactic acid accumulation in the blood for the user (e.g., an athlete) having physical strength higher than the standard and with a high rate of sugar burning exercise and the exercise can be determined as exercise that the athlete can continue for one hour or more. Therefore, the informing unit 230 informs the user that the exercise is, for example, “relatively light exercise that can be continued for one hour or more”.

As a result of collating the pulse rate during exercise of the user with the lactate level information data table 250, when the pulse rate during exercise exceeds the AT or VT threshold, the exercise is considered to be exercise with a high load in which the lactic acid accumulation in the blood suddenly increases and that even the athlete cannot continue for a long time. The informing unit 230 informs the user that the exercise is, for example, exercise with a high load with which the athlete can achieve improvement of a level such as further record improvement or inspires the user with effective sound or the like.

As explained above, with the exercise-effect determining method according to this modification, as in the first embodiment, it is possible to perform appropriate exercise evaluation for each user with respect to exercise loads in a wide range of intensity by performing the exercise evaluation using the work thresholds such as the AeT, the AT, and the VT serving as the lactate level information corresponding to an exercise load of low intensity and exercise loads of medium intensity to high intensity of each user.

Since the exercise effect determination is performed using the lactate level information based on the relation between the pulse rate and the exhalation gas (the respiration quotient), in particular, it is possible to more accurately perform discrimination of exercise with a high rate of fat burning exercise (aerobic exercise) and exercise with a high rate of sugar burning exercise (anaerobic exercise). For example, it is possible to more properly determine discrimination of exercise suitable for diet and exercise suitable for physical strength reinforcement, a warning that exercise intensity is too high and dangerous, and the like and inform the user of the discrimination, the warning, and the like.

### Second Embodiment

A second embodiment of the invention is explained with reference to the drawings.

A biological-information measuring device according to the second embodiment is a heart-rate monitoring device that is worn on an organism (e.g., a human body), biological information of which is measured, and measures biological information such as a pulse (a heart rate) like the wearable device 200 included in the system (the exercise-effect determining system) 101 in the first embodiment. Note that, in the figures referred to below, dimensions and ratios of components are sometimes shown different from those of actual components as appropriate in order to show the components in recognizable sizes on the figures.

First, before explaining a heart-rate monitoring device 1020 functioning as a wearable device (the biological-information measuring device) according to the second embodiment, an example in the past of the heart-rate monitoring device functioning as the biological-information measuring device according to the second embodiment is explained with reference to FIG. 9.

FIG. 9 is a sectional view showing the heart-rate monitoring device 1010 functioning as the biological-information measuring device in the example in the past that measures physiological parameters (biological information) of a user (a subject; in the figure, an arm of the user is shown) wearing the heart-rate monitoring device. The heart-rate monitoring device 1010 includes a sensor 1012 that measures a heart rate serving as at least one physiological parameter of the user and a case 1014 that houses the sensor 1012. The heart-rate monitoring device 1010 is worn on an arm 1 of the user by a fixing section 1016 (e.g., a band).

The sensor 1012 is a heart-rate monitoring sensor including a light emitting element 1121 functioning as a light emitter and a light receiving element 1122 functioning as a light receiver, which are two sensor elements, and for measuring or monitoring a heart rate. However, the sensor 1012 may be a senor that measures one or more physiological parameters (e.g., a heart rate, a blood pressure, an exhalation amount, skin conductivity, and skin humidity). When the case 1014 includes a housing of a band type, for example, the sensor 1012 can be used as, for example, a wristwatch-type monitoring device used in sports. Note that the shape of the case 1014 may be any shape as long as the sensor 1012 can be held in a desired position mainly with respect to the user (in the figure, the arm 1 of the user). The case 1014 may be able to optionally house other devices such as a battery, a processing unit, a display, and a user interface.

The biological-information measuring device of the example in the past is the heart-rate monitoring device 1010 for monitoring a heart rate of the user. The sensor 1012 is an optical sensor including the light emitting element 1121 and the light receiving element 1122. An optical heart rate monitor including the optical sensor depends on the light emitting element 1121 (usually, an LED is used) functioning as a light source that radiates light on skin. The light radiated on the skin from the light emitting element 1121 is partially absorbed by blood flowing in blood vessels under the skin. However, the remaining light is reflected to exit the skin. The reflected light is captured by the light receiving element 1122 (usually, a photodiode is used). A light reception signal from the light receiving element 1122 is a signal including information equivalent to an amount of blood flowing in the blood vessels. The amount of blood flowing in the blood vessels changes according to the pulsation of the heart. In this way, the signal on the light receiving element 1122 changes according to the beat of the heart. That is, the change in the signal of the light receiving element 1122 is equivalent to a pulse of a heart rate. The number of beats (i.e., a heart rate) per one minute of the heart is obtained by counting the number of pulses per unit time (e.g., per 10 seconds).

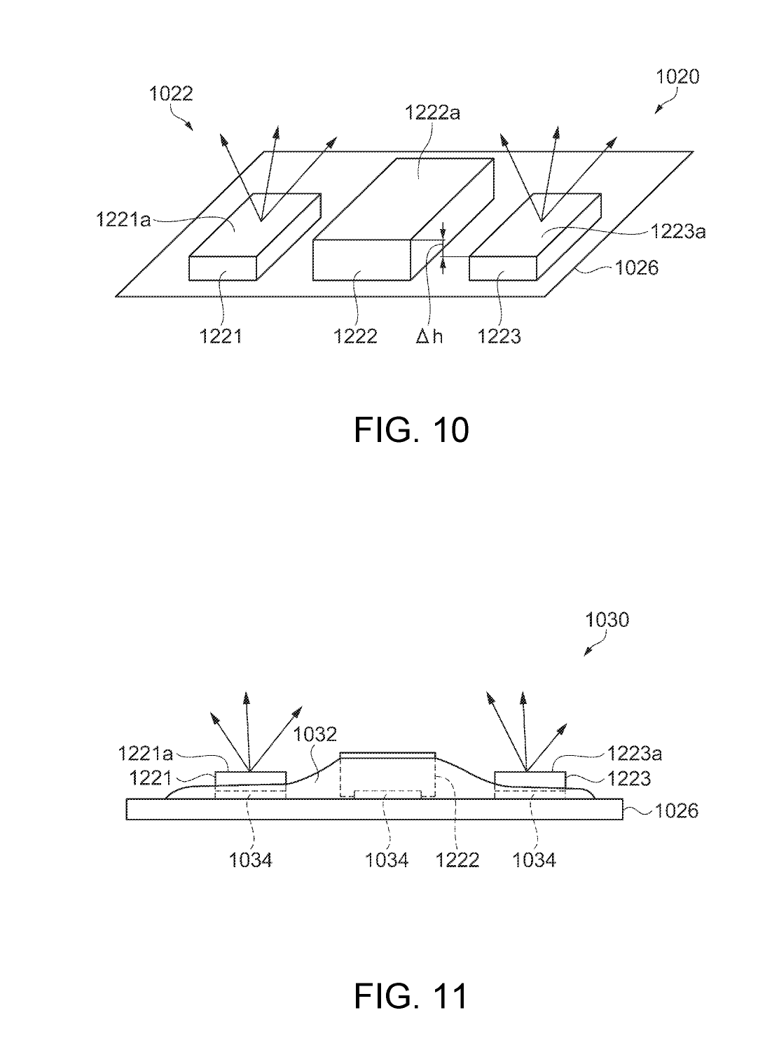

The heart-rate monitoring device 1020 functioning as the wearable device (the biological-information measuring device) according to the second embodiment is explained below with reference to FIG. 10. FIG. 10 is a perspective view showing the heart-rate monitoring device according to the second embodiment. Although not shown in FIG. 10, as in the first embodiment, the heart-rate monitoring device 1020 according to the second embodiment is worn on an arm of the user by a fixing section such as a band.

In the heart-rate monitoring device 1020 according to the second embodiment, light emitting elements 1221 and 1223 functioning as a plurality of (in this example, two) light emitters and a light receiving element 1222 functioning as one light receiver are disposed in a row. Specifically, the heart-rate monitoring device 1020 includes a sensor 1022 including at least two sensor elements (in this example, the two light emitting elements 1221 and 1223 functioning as a first light emitter and a second light emitter and the light receiving element 1222 functioning as a light receiver are used as three sensor elements). Note that, although not shown in the figure, it is desirable that the heart-rate monitoring device 1020 includes a first frame and a second frame provided between the light receiving element 1222 and the light emitting element 1221 and between the light receiving element 1222 and the light emitting element 1223 to surround each of the light receiving element and the light emitting elements.

The light receiving element 1222 functioning as the light receiver is disposed between the two light emitting elements 1221 and 1223 functioning as the first light emitter and the second light emitter. The two light emitting elements 1221 and 1223 functioning as the first light emitter and the second light emitter are disposed in positions symmetrical with respect to an imaginary line passing the center of the light receiving element 1222 functioning as the light receiver. By disposing the light emitting elements 1221 and 1223 and the light receiving element 1222 in this way, a dead space decreases. It is possible to achieve space saving. Lights emitted from the first light emitter and the second light emitter present in the symmetrical positions concentrate on the light receiver. Therefore, it is possible to perform more accurate detection.

The sensor elements detect sensor signals. The sensor 1022 includes an optical sensor including the light emitting elements 1221 and 1223, in which two LEDs for emitting lights to the skin of the user are used, and at least one light receiving element 1222 (a photodiode) for receiving light reflected from the skin. Further, the heart-rate monitoring device 1020 includes a case or a housing (not shown in the figure). The case or the housing may be similar to or the same as the case 1014 shown in FIG. 9 or may be similar to or the same as the case section 30 in the first embodiment.

The sensor 1022 is carried on the entire surface of a carrier (a substrate) 1026. A component including the carrier (the substrate) 1026 and the sensor 1022 carried on the carrier (the substrate) 1026 is equivalent to a biological-information measuring module. Note that the same applies in third to fifth embodiments explained below. Lights emitted from the light emitting elements 1221 and 1223 are reflected without being absorbed by the skin and the like and can directly reach the light receiving element 1222. In the heart-rate monitoring device 1020, the distance between the carrier 1026 and upper surfaces 1221a and 1223a of the light emitting elements 1221 and 1223 is smaller than the distance between the carrier 1026 and an upper surface 1222a of the light receiving element 1222. That is, a difference between the distance between the carrier 1026 and the upper surfaces 1221a and 1223a of the light emitting elements 1221 and 1223 and the distance between the carrier 1026 and the upper surface 1222a of the light receiving element 1222 is Δh. The light receiving element 1222 receives light from the upper surface 1222a, which is the uppermost layer of the light receiving element 1222. With these components, there is an effect that most of the lights emitted from the light emitting elements 1221 and 1223 travel to the skin and reflected light is directly made incident on the light receiving element 1222 without intervention of an air layer and the like. In other words, since the light receiving element 1222 adheres to the skin, a gap is less easily formed between the upper surface (the light receiving surface) 1222a of the light receiving element 1222 and the skin. Consequently, it is possible to suppress light such as external light, which is a noise source, from being made incident on the upper surface 1222a. Lights from the light emitting elements 1221 and 1223 not passing through the skin, for example, lights directly made incident on the light receiving element 1222 from the light emitting elements 1221 and 1223 cannot reach the upper surface 1222a of the light receiving element 1222.

### Third Embodiment

A heart-rate monitoring device 1030 functioning as a wearable device (a biological-information measuring device) according to a third embodiment is explained with reference to FIG. 11. FIG. 11 is a sectional view showing the heart-rate monitoring device according to the third embodiment. Note that, although not shown in FIG. 11, as in the first embodiment, the heart-rate monitoring device 1030 according to the third embodiment is worn on an arm of a user by a fixing section such as a band.

As shown in FIG. 11, electric connection terminals 1034 of the light emitting elements 1221 and 1223 functioning as the light emitters and the light receiving element 1222 functioning as the light receiver desirably have to be covered with an insulative material (e.g., epoxy resin) 1032 for protection of electric elements. The insulative material 1032 can be configured not to cover the light emitting elements 1221 and 1223 and the light receiving element 1222. Specifically, a region between the light emitting element 1221 and the light receiving element 1222 and a region between the light emitting element 1223 and the light receiving element 1222 can be filled with the insulative material 1032. In other words, at least the upper surface 1222a of the light receiving element 1222 and the upper surfaces 1221a and 1223a of the light emitting elements 1221 and 1223 can be configured not to be covered with the insulative material 1032. By adopting such a configuration, it is possible to suppress interference by air gaps between the skin and the light emitting elements 1221 and 1223. Further, the insulative material 1032 may be configured to cover the upper surfaces 1221a and 1223a of the light emitting elements 1221 and 1223 and the upper surface 1222a of the light receiving element 1222. By adopting such a configuration, it is possible to protect the upper surface 1222a of the light receiving element 1222 in contact with the skin and the upper surfaces 1221a and 1223a of the light emitting elements 1221 and 1223. Therefore, it is possible to prevent damage to the upper surface 1222a of the light receiving element 1222 and the upper surfaces 1221a and 1223a of the light emitting elements 1221 and 1223. In this case, the insulative material 1032 can also be regarded as a protection film.

In the heart-rate monitoring device 1030 according to the third embodiment, as a generally possible example, the insulative material 1032 including epoxy resin is provided. In FIG. 11, the insulative material 1032 is disposed not to cover the upper surfaces 1221a and 1223a of the light emitting elements 1221 and 1223 and protects the electric connection terminals 1034. Lights emitted from the light emitting elements 1221 and 1223 are indicated by arrows.

In this way, the insulative material 1032 is disposed as small as possible not to prevent the correct function of the heart-rate monitoring device 1030, whereby the electric connection terminals 1034 of the light emitting elements 1221 and 1223 and the light receiving element 1222 are protected. Consequently, the heart-rate monitoring device 1030 can be further improved. Note that, although not shown in the figure, it is more suitable that the heart-rate monitoring device 1030 includes a first frame and a second frame provided between the light receiving element 1222 and the light emitting element 1221 and between the light receiving element 1222 and the light emitting element 1223 to surround each of the light receiving element and the light emitting elements.

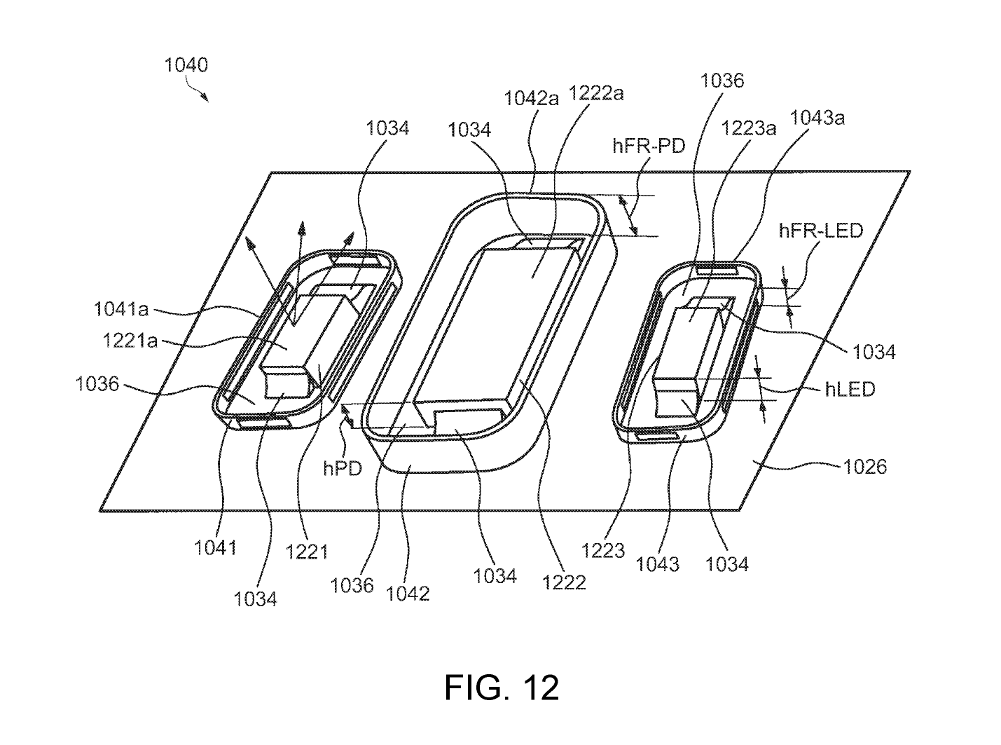

Note that it is more suitable to adopt, instead of the configuration in which the epoxy resin is injected in the third embodiment, a heart-rate monitoring device 1040 functioning as a biological-information measuring device according to a fourth embodiment shown in FIG. 12.

### Fourth Embodiment

A heart-rate monitoring device 1040 functioning as a wearable device (a biological-information measuring device) according to a fourth embodiment is explained with reference to FIG. 12. FIG. 12 is a perspective view showing the heart-rate monitoring device according to the fourth embodiment. Note that, although not shown in FIG. 12, as in the first embodiment, the heart-rate monitoring device 1040 according to the fourth embodiment is worn on an arm of a user by a fixing section such as a band.

In the heart-rate monitoring device 1040 according to the fourth embodiment, crated frames 1041, 1042, and 1043 are disposed. The frames 1041, 1042, and 1043 are disposed around the light emitting elements 1221 and 1223 functioning as the light emitters and the light receiving element 1222 functioning as the light receiver. Gaps 1036 between the frames 1041, 1042, and 1043 and the light emitting elements 1221 and 1223 and the light receiving element 1222 are formed. An insulative material (not shown in FIG. 12) is injected using the frames 1041, 1042, and 1043 as guides and covers the electric connection terminals 1034 of the light emitting elements 1221 and 1223 and the light receiving element 1222.

In an example explained in the fourth embodiment, the light emitting elements 1221 and 1223 and the light receiving element 1222 are surrounded by the respective frames 1041, 1042, and 1043. Note that, as another example, all of the frames 1041, 1042, and 1043 may be joined to one another. Alternatively, all the sensor elements may be surrounded by an integral frame. Note that the frames 1041, 1042, and 1043 can be used as light blocking walls, which are an example of a light blocker. By using the frames 1041, 1042, and 1043 as the light blocking walls, it is possible to prevent lights emitted from the light emitting elements 1221 and 1223 from being directly entering the light receiving element 1222.

As an improvement for preventing the function of the heart-rate monitoring device 1040 from being affected, upper edges 1041a and 1043a of the frames 1041 and 1043 around the light emitting elements 1221 and 1223 are desirably lower than the upper surfaces 1221a and 1223a of the light emitting elements 1221 and 1223. In other words, a distance hFR-LED between the upper edges 1041a and 1043a of the individual frames 1041 and 1043 and the carrier 1026 is the same as or smaller than a distance hLED between the upper surfaces 1221a and 1223a of the light emitting elements 1221 and 1223 surrounded by the individual frames 1041 and 1043 and the carrier 1026 (hFR−LED≧LED).

Desirably, a difference between the distance hLED between the upper surfaces 1221a and 1223a of the light emitting elements 1221 and 1223 and the carrier 1026 and the distance hFR-LED between the upper edges 1041a and 1043a of the frames 1041 and 1043 and the carrier 1026 is set in a range of 0.1 mm to 0.8 mm. Note that, more desirably, the difference between the distance hLED between the upper surfaces 1221a and 1223a of the light emitting elements 1221 and 1223 and the carrier 1026 and the distance hFR-LED between the upper edges 1041a and 1043a of the frames 1041 and 1043 and the carrier 1026 is set in a range of 0.2 mm to 0.5 mm.

The upper edge 1042a of the frame (a receiver frame) 1042 around the light receiving element 1222 is desirably higher than the upper surface 1222a of the light receiving element 1222 functioning as the light receiver. In other words, a distance hFR-PD between the upper edge 1042a of the frame 1042 and the carrier 1026 is larger than a distance hPD between the upper surface 1222a of the light receiving element 1222 surrounded by the frame 1042 and the carrier 1026 (hFR−PD>hPD).

Desirably, a difference between the distance hPD between the upper surface 1222a of the light receiving element 1222 and the carrier 1026 and the distance hFR-PD between the upper edge 1042a of the frame 1042 and the carrier 1026 is set in a range of 0 mm to 0.5 mm. Note that, more desirably, the difference between the distance hPD between the upper surface 1222a of the light receiving element 1222 and the carrier 1026 and the distance hFR-PD between the upper edge 1042a of the frame 1042 and the carrier 1026 is set in a range of 0.1 mm to 0.2 mm.

Further, the distance hFR-PD between the upper edge 1042a of the frame 1042 and the carrier 1026 is larger than the distance hLED between the upper surfaces 1221a and 1223a of the light emitting elements 1221 and 1223 and the carrier 1026 (hFR−PD>hLED).

Note that, for example, when the light receiving element 1222 and the light emitting elements 1221 and 1223 are close to each other, only one frame wall maybe present between the light receiving element 1222 and the light emitting elements 1221 and 1223. This is sometimes caused because of manufacturing easiness. When the one frame wall is a case, frame walls of both of frames coincide with each other in the light receiving element 1222 and the light emitting elements 1221 and 1223. This means that the frame walls of the light emitting elements 1221 and 1223 are higher. Specifically, the frame walls on a side where the light receiving element 1222 is present in the frames 1041 and 1043 surrounding the light emitting elements 1221 and 1223 are high. The other frame walls are lower than the upper surfaces 1221a and 1223a of the light emitting elements 1221 and 1223.

Further, instead of the frames 1041, 1042, and 1043, a first wall section may be provided between the light receiving element 1222 and the light emitting element 1221 or the light emitting element 1223 and a second wall section may be provided on the outer side of the light emitting elements 1221 and 1223, that is, on the opposite side of the first wall section with respect to the light receiving element 1222.

In such a configuration, the distance between the carrier 1026 and the upper surface of the first wall section may be set larger than the distance between the carrier 1026 and the upper surface of the second wall section. By adopting such a configuration, compared with when the light emitting elements and the light receiving element are surrounded as shown in FIG. 12, it is possible to realize the function of the frames with fewer members.

Note that, by using the frames 1041 and 1043 and the frame 1042 as in the fourth embodiment, it is possible to prevent an injected insulative material such as epoxy resin from flowing out. Creating an additional structure to partition the insulative material such as the epoxy resin in this way is an option for enabling high mass productivity. Note that the frames 1041 and 1043 and the frame 1042 may be formed of a material same as the material of the carrier 1026. For example, the frames may be formed by injection molding using epoxy-based resin or polycarbonate-based resin.

As explained above, the insulative material 1032 (see FIG. 11) protects the electric connection terminals 1034 of the sensor elements (the light emitting elements 1221 and 1223 and the light receiving element 1222). However, the electric connection terminals 1034 have to be further in contact with additional electronic devices (e.g., a driver, a detection electronics, a processor, and a power supply), which are other element. This means that some electric connection to the additional electronic devices is present in the carrier 1026 (which may be a printed board (PCB)). The structure of the heart-rate monitoring device according to this embodiment can be applied to not only a measuring device of a heart rate but also a measuring device of a pulse wave and a pulse.

### Fifth Embodiment

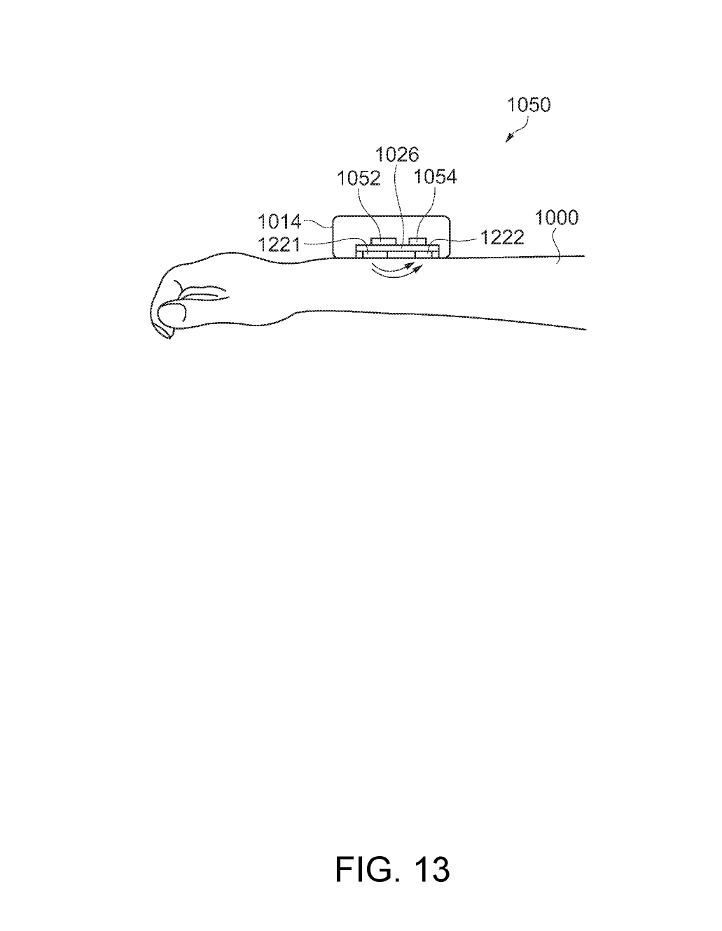

A heart-rate monitoring device 1050 functioning as a wearable device (a biological-information measuring device) according to a fifth embodiment is explained with reference to FIG. 13. FIG. 13 is a sectional view showing the heart-rate monitoring device according to the fifth embodiment. Note that, although not shown in FIG. 13, as in the first embodiment, the heart-rate monitoring device 1050 according to the fifth embodiment is worn on an arm of a user by a fixing section such as a band.

The heart-rate monitoring device 1050 according to the fifth embodiment includes the additional electronic devices (e.g., a processor 1052 and a driver 1054) explained above. An external electric connection terminal (not shown in the figure) is not disposed on the carrier 1026 on which the sensor elements (the light emitting element 1221 functioning as the light emitter and the light receiving element 1222 functioning as the light receiver) are disposed. That is, the additional electronic devices are disposed on a carrier or a substrate different from a carrier or a substrate on which the sensor elements are disposed. By adopting such a configuration, it is possible to mount necessary additional electronic devices on the heart-rate monitoring device 1050 while maintaining satisfactory contact of the skin and the sensor elements (the light emitting element 1221 and the light receiving element 1222). For example, the external electric connection terminal can be disposed on a side surface of the carrier 1026.

As explained above, different kinds of sensors can be used in the biological-information measuring device according to the invention. For example, when the light receiving element 1222 is an electric sensor, two skin conductance electrodes (e.g., the sensor elements (the light emitting element 1221 and the light receiving element 1222 shown in FIG. 10)) set in contact with the skin of the user to measure the conductivity of the user are covered with the skin. Note that further two or more kinds of sensors can be used in the biological-information measuring device of this type. Further, the number of sensor elements may be any number.

A method of manufacturing a biological-information measuring device that measures proposed physiological parameters in the second to fifth embodiments is explained.

First, in a first step S1, the sensor 1022 including at least two sensor elements (the light emitting element 1221 and the light receiving element 1222) for detecting a sensor signal is disposed on the carrier 1026. Subsequently, in a second step S2, an electric contact of the sensor elements is formed on the carrier 1026. In a third step S3, one or more frames 1041 and 1042 are formed on the carrier 1026 around the sensor 1022 and/or the respective sensor elements (the light emitting element 1221 and the light receiving element 1222). In a fourth step S4, the insulative material 1032 is injected and filled in regions that do not cover the upper surfaces 1221a and 1222a of the sensor elements (the light emitting element 1221 and the light receiving element 1222) provided on the carrier 1026 and are surrounded by the respective frames 1041 and 1042.

According to the second to fifth embodiments, there is proposed a method of achieving protection of the electric contact without adversely affecting the performance of the biological-information measuring device. The biological-information measuring device is formed by a method of keeping the performance of the sensors. For example, at least one of the frames 1041 and 1043 prevents the positions of the sensors with respect to the skin from shifting. Further, at least one of the frames 1041 and 1043 can be useful for preventing emitted direct light from being made incident on the light receiving element 1222. Desirably, the height of the frames 1041 and 1043 around the light emitting elements 1221 and 1223 on a side facing the light receiving element 1222 has to be smaller than the height of the upper surfaces 1221a and 1223a of the light emitting elements 1221 and 1223. In addition, the frame 1042 around the light receiving element 1222 may be higher than the upper surface 1222a of the light receiving element 1222.

The embodiments of the invention devised by the inventor are specifically explained above. However, the invention is not limited to the embodiments explained above. It is possible to add variously changes without departing from the spirit of the invention.

For example, in the exercise-effect determining method in the embodiment, the exercise start of the user is automatically determined on the basis of the body motion signal acquired by the body motion sensor 220. However, it is also possible to cause the user to manually input, using a button or the like, timing of a pulse wave signal measurement start to calculate a pulse rate during exercise.

Not that the embodiments and the modifications of the invention are explained above in detail. However, those skilled in the art could easily understand that many modifications are possible without substantially departing from the new matters and the effects of the invention. Therefore, all such modifications are deemed to be included in the scope of the invention. For example, terms described together with broader or synonymous different terms at least once in the specification or the drawings can be replaced with the different terms in any place in the specification or the drawings. The configurations and the operation of the exercise-effect determining system and the wearable device (the biological-information detecting device) are not limited to the configurations and the operations explained in the embodiments. Various modified implementations of the configurations and the operations are possible.

What is claimed is:

1. An exercise-effect determining method comprising:
acquiring, from a pulse wave sensor and a body motion sensor, a pulse wave signal and a body motion signal of a user at time when the user is performing predetermined exercise;
a determiner determining, on the basis of a pulse rate during exercise calculated on the basis of the pulse wave signal and the body motion signal and lactate level information representing a relation between a pulse rate and a blood lactate level of the user acquired in advance, a degree of the predetermined exercise contributing to maintenance or reinforcement of physical strength of the user.

2. The exercise-effect determining method according to claim 1, wherein the lactate level information includes one of a lactate threshold and an onset of blood lactate accumulation.

3. The exercise-effect determining method according to claim 1, wherein the lactate level information includes at least any one of an aerobic threshold, an anaerobic threshold, and a ventilation threshold.

4. The exercise-effect determining method according to claim 2, wherein the user is capable of setting the lactate level information according to a purpose.

5. The exercise-effect determining method according to claim 3, wherein the user is capable of setting the lactate level information according to a purpose.

6. The exercise-effect determining method according to claim 1, wherein an exercise-effect determination result is informed from the determiner to the user.

7. An exercise-effect determining system comprising:
a lactate level information data table in which lactate level information representing a relation between a pulse rate and a blood lactate value of a user is stored;
a pulse wave measurer configured to measure a pulse wave signal of the user at time when the user is performing predetermined exercise;
a body motion detector configured to detect a body motion signal of the user at the time when the user is performing the predetermined exercise;
a pulse rate calculator configured to calculate a pulse rate during exercise on the basis of the pulse wave signal and the body motion signal; and
an exercise effect determiner configured to obtain, on the basis of a comparison result of the lactate level information of the lactate level information data table and the lactate level information corresponding to the pulse rate during exercise, an exercise-effect determination result of determination of a degree of exercise intensity of the predetermined exercise contributing to maintenance or reinforcement of physical strength of the user.

8. The exercise-effect determining system according to claim 7, wherein
the lactate level information includes a plurality of kinds of indexes, and
the exercise-effect determining system further comprises a determination mode setter with which the user is capable of setting anyone of the plurality of kinds of indexes as the lactate level information according to a purpose.

9. The exercise-effect determining system according to claim 8, wherein the plurality of kinds of indexes include one of a lactate threshold and an onset of blood lactate accumulation.

10. The exercise-effect determining system according to claim 8, wherein the plurality of kinds of indexes include at least anyone of an aerobic threshold, an anaerobic threshold, and a ventilation threshold.

11. The exercise-effect determining system according to claim 7, further comprising an informer configured to inform the user of the exercise-effect determination result.

12. An exercise-effect determining device comprising:
an acquirer configured to acquire a pulse wave signal and a body motion signal of a user;
a storage configured to store lactate level information representing a relation between a pulse rate and a blood lactate level of the user; and
a determiner configured to determine, on the basis of a pulse rate during exercise calculated on the pulse wave signal and the body motion signal and the lactate level information, a degree of exercise carried out by the user contributing to maintenance or reinforcement of physical strength of the user.

13. The exercise-effect determining device according to claim 12, wherein
the lactate level information includes a plurality of kinds of indexes, and
the plurality of kinds of indexes include at least one of a lactate threshold and an onset of blood lactate accumulation.

14. The exercise-effect determining device according to claim 13, wherein the determiner includes a first mode for determining, on the basis of the lactate threshold, a degree of exercise carried out by the user contributing to maintenance or reinforcement of physical strength of the user and a second mode for determining, on the basis of the onset of blood lactate accumulation, a degree of exercise carried out by the user contributing to maintenance or reinforcement of the physical strength of the user.

15. The exercise-effect determining device according to claim 12, wherein
the lactate level information includes a plurality of kinds of indexes, and
the plurality of kinds of indexes include any one of an aerobic threshold, an anaerobic threshold, and a ventilation threshold.

16. The exercise-effect determining device according to claim 12, further comprising an informer configured to inform the user of an exercise-effect determination result in the determiner.

17. The exercise-effect determining device according to claim 12, further comprising:
a pulse wave sensor configured to output a pulse wave signal of the user; and
a body motion sensor configured to output a body motion signal of the user.
